(19) United States
(12) Patent Application Publication
(10) Pub. No.: US 2008/0233242 A1
(43) Pub. Date: Sep. 25, 2008

(54) Antioxidant of Bamboo Leaves and Its Uses

(76) Inventors: Ying Zhang, Hang Zhou (CN); Xiaoqin Wu, Hang Zhou (CN); Zhuoyu Yu, Hang Zhou (CN); Dingding Luo, Hang Zhou (CN); Boyi Lu, Hang Zhou (CN); Yu Zhang, Hang Zhou (CN)

Correspondence Address: YUAN QING JIANG, P.O. BOX 61214, PALO ALTO, CA, 94306, US

(21) Appl. No.: 10/575,007

(22) PCT Filed: Oct. 8, 2004
(86) PCT No.: PCT/CH04/01133

§ 371 Date: Mar. 14, 2008

(30) Foreign Application Priority Data
Oct. 8, 2003 (CN) 200310107871.5

(51) Int. Cl. A23L 3/3463; A23L 1/302; C07D 311/30; C07D 311/08; C07C 69/732; C07C 59/52

(52) U.S. Cl. 426/72; 426/545; 426/546; 549/403; 549/399; 560/61; 562/465

(57) ABSTRACT

The present invention discloses the composition of antioxidant of bamboo leaves (AOB) and its use. The purpose of present invention is to provide a new food additive which is natural, nutritional, and muti-functional, and which is of rich resources, safety, good effect, and low cost. AOB is yellow or brown powders or particles obtained from bamboo leaves, wherein the main antioxidative components include flavones, lactones, phenolic acids. AOB can either inhibit lipid autoxidation chain reaction, or chelate transitional metal ions, and can be used as primary and second antioxidant. AOB can eliminate nitrite and inhibit the synthesis of N-nitrosamine, and has anti-bacteria, bacteriostatic, deodorizing, aroma enhancing etc. functions. AOB can be commonly used in oil-containing food, meat product, fishery product, expanded food etc. food systems.

### TECHNICAL FIELD

The present invention relates to the field of food additives, and particularly to an antioxidant of bamboo leaves (AOB) and the use thereof. The present invention provides a natural, multi-functional food additive with rich resources, good safety, high quality, and low cost. Specifically, this invention provides the chemical composition, physical and chemical properties, and antioxidation of the AOB extracted from Bamboo Leaves (Herba Lophatheri), and the use thereof in edible oil, oil-containing food, meat products, aquatic products, fruit juice, soft drinks, brewed wine, milk products, condiments, puffed food, and cakes, etc.

### BACKGROUND ART

Food industry is associated with national economy and people's livelihood, and plays an extremely significant role in economic field of various countries. The three essential factors to restrict the development of modern food industry are raw material, process and equipment, and food additive which is the most active and positive one among the three factors.

Presently, there is a great gap between the domestic food additive industry and the advanced level in foreign countries in quality, safety, and amount. Recently, the additive industry in our country actively advocates the “Natural, Nutritional, and Multi-functional” policy, and this is in accordance with the international trend of going back to nature. China Green Food Development Center prescribes in various standards issued in September 1999 related to green food that,: only natural additives are permitted to be used in AA green food, any chemical synthetic additive is prohibited in its production process, and any chemical synthetic pesticide and synthetic food additive should not be detected in its finished products. With vast area and rich resources, our country has the tradition of obtaining food and drug from the same source. Therefore, our country has a unique advantage in developing natural, nutritional, and multi-functional food additives. As food additive is of multiple types, stress in developing these food additives should be laid on new types with good quality, high safety, low prices and without peculiar smell. The development of natural food additives has become an irreversible international trend.

The antioxidant is the weakest link in product structure of food additive industry in our country. Using antioxidants to prevent food oxidation is the commonest and very effective method in food storage. The antioxidant generally includes two types, i.e. oil-soluble and water-soluble types. The former includes natural vitamin E (VE), artificial propyl gallate, ascorbates, butylated hydroxyanisole (BHA), dibutylated hydroxytoluene (BHT), and tert-butyl hydroquinone (TBHQ), etc.; and the latter includes ascorbic acid (vitamin C, VC), iso-ascorbic acid (iso-vitamin C) and iso-ascorbates, phytic acid, and tea polyphenol, etc. The synthetic antioxidants such as BHA and BHT, which are used much more, have some safety problems. Animal experiments showed that they have some toxic and carcinogenic effects, therefore the uses thereof have been restricted in most developed countries. For example, BHA is carcinogenic to rat's proventriculus, and in 1982 it was limited to use only in palm oil in Japan; BHT inhibits human respiratory enzyme and increases hepatic microsomal enzyme activity, thus its use was once prohibited in America; animal experiments showed that TBHA has mutagenic potential, and thus it is not approved in European countries and Japan; and PG is of high price and of narrow use range. Moreover, synthetic food additives are limited to use due to human doubts and repulsive psychology about them.

Presently, there are 47 commercial products of natural antioxidant in foreign countries, wherein the rosemary extract has a stronger antioxidation than BHA and BHT, and others include salvia extract, licorice antioxidants, tea polyphenol, ellagic acid, and sunflower seeds extract, etc. Although rosemarinic acid and salviol have stronger antioxidation and are used worldwide, their uses are greatly limited due to three reasons: very high requirement for geo-environment, low output and high price of the raw material. In our country, the natural antioxidants approved (listed in GB-2760) mainly include tea polyphenol, phytic acid (phytate sodium), liquorice antioxidant, and phospholipid, etc. With advantages of safety and no toxicity, natural antioxidants are welcome and have held the limelight in current development.

The development of food additives is focused on natural, nutritional, multi-functional, safe, and reliable additives. It is the developmental trend in future food industry to replace synthetic antioxidants with natural edible antioxidants, and it is a top priority to develop practical, highly effective, and non-expensive natural antioxidants with characteristics of local resources and independent intellectual property.

Our country is always known as the “Kingdom of Bamboo”, provided with extremely rich bamboo resources and with bamboo culture of long standing. In the domain of our country, there are more than 40 families of bamboos with more than 400 species of bamboos, approximately 4 million ha of bamboo forest area. According to incomplete statistic data, there are more than 100 million people in our country who make their lives by bamboo forest or by processing bamboo products. Bamboo is a significant part of forest resources, not only with high economic values but also with extensive ecological and social benefits. Bamboo is being paid more and more attention for their particular biological, ecological characteristics and multiple-use. They play a more and more important role in the sustainable development strategy of China.

Bamboo leaf (Herba Lophatheri) has been widely used in folks of our country for a long history, both in food and drug fields. It is a famous drug to clear away heat-evil and to expel superficial evils. Herba Lophatheri was initially recorded in “Other Records”, and was listed at middle level, with description of “severe cold without toxicity, indicated in phlegm-heat in the chest, and cough and belching”. In addition, according to records of Big Dictionary of Traditional Chinese Drugs, Herba Lophatheri has the action of: clearing away heat, quenching fire, promoting the production of body fluid, and promoting diuresis. Materia Medica in Food Therapy recorded: “arrest cough, and remove excess heat; indicated in phlegm with dyspepsia and inflammation of the throat.” Doctors and pharmacists in various dynasties kept description on the effect of Herba Lophatheria as food and drug, Jia Suoxue recorded in Interpretation of Traditional Chinese Drugs: “Herba Lophatheri is fragrant and cool to heart; slightly bitter, cold, and heat; odor and taste is aromatic.” Holy Prescriptions for Universal Relief recorded a diet therapy prescription: “Herba Lophatheri is indicated in pediatric heart wind and heat and absent-mindedness: Herba Lophatheri, 60 g; Japonica Rice, adequate amount; Herba Artemisiae Scopariae, 15 g; make them into conjee and eat it. In 1998, Herba Lophatheri was approved and listed in natural substance as food and drug.

The investigation and development of active ingredients of Herba Lophatheri in our country are at the leading level in the world. The bamboo leaf (Herba Lophatheri) extract is a preparation of vegetable flavonoid developed in 1990s by Zhang Ying et al. The patents for invention for this extract, i.e. “A Health Beer Supplemented with Lophatherum Flavones (ZL 98 1 04563.4)” and “Method for Producing Extract or Powder of Flavonoids from Bamboo Leaves (ZL 98 1 04564.2)”, are issued by the Patent Bureau of China in 2000 and 2001, respectively. Lots of studies have demonstrated that, Lophatherum Flavones have excellent biological effects of anti-free radicals, anti-oxidation, anti-aging, antimicrobial, anti-virus, as well as protecting cardiovascular and cerebrovascular systems, and preventing and treating senile degenerative diseases, etc. Recently, it has cut a figure in the field of functional food and medical health food, with its rich resources, specific functional factors, convincing safety, highly effective and stable preparation quality, and pure, fresh, sweet, and fragrant bamboo taste (Zhang Ying, A Natural and Functional Supplement—Lophatherum Extract, Fine and Special Chemical Substances, 2002, 10(7):20-22).

The main functional components of Lophatherum Extract are glycoside flavones, main components of which are carbonide flavones. The four main carbonide flavones of Herba Lophatheri are orientin, homoorientin, vitexin, and isovitexin. Compared with glycoside flavones, carbonide flavones have the following outstanding advantages: (1) stable structure, difficult to be decomposed; (2) deeply dispersed into the site of focus, and directly exerting therapeutic effects; (3) increased hydrophilicity, favorable to development of drugs, foods, and cosmetics. Carbonide flavones have been paid with attention by international academic circle, and this field is the newest edge of study. Presently, flavones of Herba Lophatheri are mainly used as medical intermediates, healthcare food materials, and nutrition intensifier of drinks and wines, etc.

However, for Lophatherum Flavones, a potential key species of plant extracts with local resources characteristics and independent intellectual property, it is far from adequacy to restrict their use in the above application fields. A basic industry of national economy with solid basis, huge capacity, stable demand, and sustainable development must be found as a support for Lophatherum Flavones. In view of the unique background of Herba Lophatheri as “food and drug from the same source”, and of the excellent quality of Lophatherum Flavones, they have vast potential of future application in food industry.

### CONTENTS OF THE INVENTION

The objective of this invention is to provide an antioxidant of bamboo leaves and the use thereof.

The chemical structures of the representative component of AOBs are as follows:

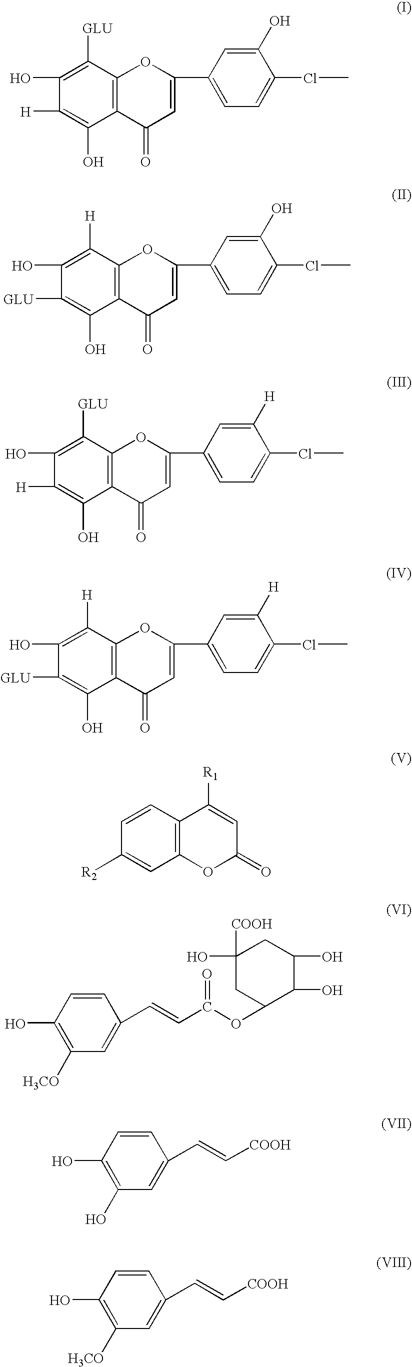

The antioxidant of bamboo leaves as a natural, nutritional, and multi-functional additive can be used in food industry.

The advantages of this invention is to provide an economical, practical antioxidant of bamboo leaves (AOB) with vast source, safety and high effectiveness, mild flavor, and stable performance, i.e. a new natural, nutritional, and multi-functional food additive with local recourses characteristics in our country. In addition to effective lipid antioxidation, its multi-functions are also characterized by antibacterial, bacteriostatic, and deodorizing effects, and by some nutritional and healthcare effects. Compared with products of the same kind, its outstanding advantages are also represented in processing meat products: it has the same antioxidation as VE, and after combined with iso-ascorbate sodium, it may greatly decrease the use amount of nitrates or nitrites and their residues, inhibit formation of N-nitrosamine, and thereby increase safety and commercial value of meat products. It may be extensively used in edible oil, oil-containing food, Chinese or Western meat products, aquatic products, fruit juice, milk products, soft drinks, brewed wine, condiments, puffed food, and cakes, etc., with vast potential in future application.

### DESCRIPTION OF THE DRAWINGS

FIG. 1 is the infrared chromatogram of AOB (tabletted with potassium bromide).

FIG. 2 is the ultraviolet chromatogram of AOB (dissolved in spectrum-pure methanol).

FIG. 3 is the high-performance liquid chromatogram of AOB.

### MODE OF CARRYING OUT THE INVENTION

The term “Antioxidant of Bamboo Leaves (AOB)”, as used herein, is a generic term for phenols obtained from leaves of Graminae, Bambusoideae, and Phyllostachys Sieb. Et Zucc. AOB according to this invention may be obtained by the patented process (Patent Number: ZL 98 1 04564.2) followed by crystallization. Said patented process is as follows specifically: Bamboo Leaves→Clean→Dry Crunch→Extract in heating reflux with ethanol-water solution→Filtrate→Concentrate in reduced pressure→Allow to stand and flocculate→Separate and remove impurities→Extract with n-butyl alcohol in scheduled steps→Concentrate in reduced pressure and recover solvent→crystallized in incubation→Centrifuge and separate crystal→Wash crystal with 95% ethanol→Dry in vacuum→AOB. On the basis of raw extracts of flavones of various bamboo leaves, the high-purity preparation may also be obtained by further using absorption-desorption, column chromatography, membrane separation, chromatographic separation, and combination of other methods.

AOB is yellow or brown-yellow powder or particles in appearance, total flavones content ≧30% (calculated on rutin through aluminum nitrate-sodium nitrite colorimetry), total lactones content ≧15% (calculated on aescine through iso-hydroximic acid colorimetry), and phenolic acids content ≧7.5% (notes: phenolic acids=total phenols−total flavones; the total phenols is determined with forint reagent reduction-colorimetry, taking p-hydroxybenzoic acid as reference).

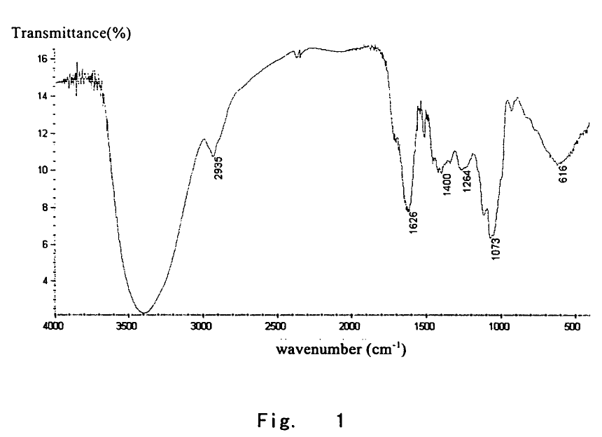

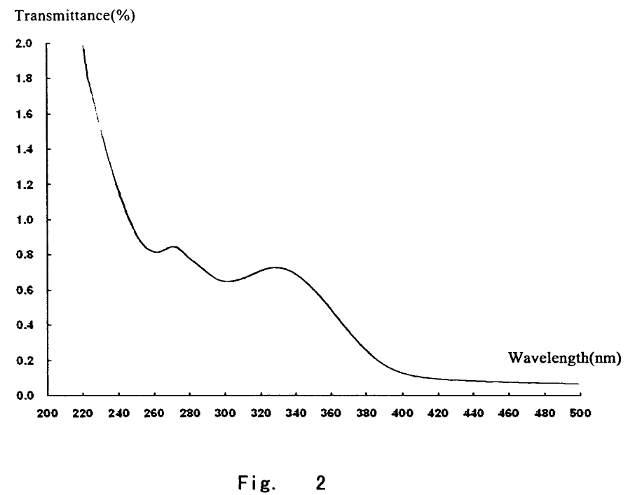

The infrared chromatogram of AOB tabletted through potassium bromide has showed that there are characteristic absorptions at or near 3400, 2935, 1626, 1079, and 616 cm⁻¹, etc. (see FIG. 1). AOB was Dissolved in spectrum-pure methanol to produce a solution which is scanned under the wavelength range of 200-600 nm: the ultraviolet chromatogram has showed that there are two key absorption peaks in the range of 240-400 nm, including a strong absorption peak at 270 nm and a secondary strong absorption at 330 nm (see FIG. 2).

Identification of AOB with chemical reagents: Dissolve the sample 0.5 g in 95% ethanol 100 mL to produce a solution, and identify the sample as follows: (1) add 1% FeCl₃ 2-3 drops to 1 mL of the solution, then deep blue or blue violet color should appear. (2) Add 1% AlCl₃-ethanol solution 2-3 drops to 1 mL of the solution, then bright yellow color should appear. Add 10 mL ether to the sample 0.5 g, extract for 30 s assisted with ultrasonic wave, and then filter. Place the filtrate 1 mL on a water bath at 70-90° C., evaporate ether to dry, and add successively 2% m-dinitrobezene (reconstituted with 95% ethanol) and 2.5 mol/L KOH (potassium hydroxide) in water, 1 ml each; slight red color should immediately appear, and rapidly become deep violet-red color after placed on the above water bath.

The newest study results in this invention have showed that, the components further separated by column chromatography and counter-flow chromatography have the antioxidatioin close or equal to that of this product, and therefore, AOB is a group of complex mixture with mutual synergetic effect, and its key antioxidant active components include flavones, lactones, and phenolc acids. The flavones are mainly carbonide flavones, with four representative compounds as follows: (I) Orientin, C₂₁H₂₀O₁₁, 448; (II) Homoorientin, C₂₁H₂₀O₁₁, 448; (III) Vitexin, C₂₁H₂₀O₁₀, 432; and (IV) Isoriextin, C₂₁H₂₀O₁₀, 432. The lactones are mainly (V) hydroxyl-coumarin and glycosides thereof. The phenolic acids are mainly derivatives of cinnamic acid, including: (VI) Chlorogenic acid, C₁₆H₁₈O₉, 354; (VII) Caffeic acid, C₉H₈O₄, 180; (VIII) Ferulic acid, C₁₀H₁₀O₄, 194.

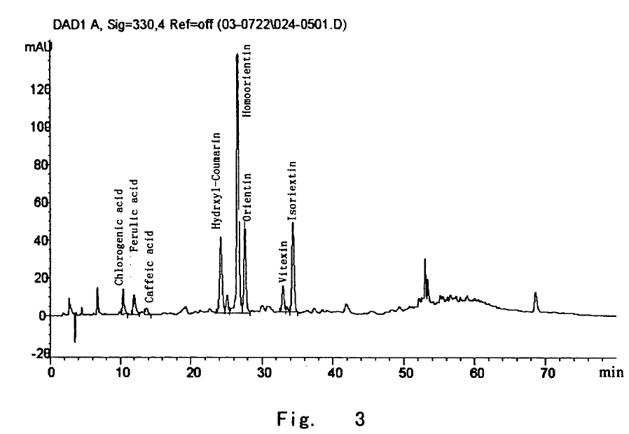

The RP-HPLC chromatogram of AOB is shown in FIG. 3. Analytical conditions are: Agilent 1100 HPLC; chromatographic column: Luna C18 column (4.60*250 mm, dp 5 m); mobile phase: acetonitrile/1% acetic acid;

Flow rate: 1 mL/min; column temperature: 40° C.; Inject volume: 30 μL. Gradient elution procedures are as follows:

| | Time (min) | | | | | | |
|---|---|---|---|---|---|---|---|
| | 0 | 12 | 20 | 35 | 50 | 70 | 80 |
| 1% acetic acid | 90% | 90% | 85% | 85% | 70% | 70% | 90% |
| acetonitrile | 10% | 10% | 15% | 15% | 30% | 30% | 10% |

AOB may block the chain reaction of lipid auto-oxidation, chelate intermediate metallic ions, and act as both primary and secondary antioxidants. With potent anti-free radical effects, it may clear away various active free oxygen-radicals (.OH, O.₂⁻, RO., and ROO., etc.). With a good antioxidant activity, it may effectively inhibit lipid peroxidation, and dramatically inhibit formation of malondialdehyde (MDA), the product of lipid peroxidation. It may effectively clear away nitrites, and block synthesis of nitrosamine, a potent carcinogenic substance. Meanwhile, it has a potent bacteriostatic action, and may inhibit Salmonella typhimurium, gram-negative bacilli, and gram-positive cocci, etc. Moreover, under some circumstances, AOB shows good effects in pigmentation, increasing fragrance, modifying taste, and removing odor, etc.

AOB has mild flavor and tastes, without any drug flavor, bitterness, or irritant smell. It has good water-solubility and stable quality. It may effectively resist against acid, heat, and enzyme, therefore suitable for many food systems. In addition to effective lipid antioxidation, its multi-functions are represented in that, it is a natural yellow pigment, and has antibacterial, bacteriostatic, deodorizing, and fragrance-increasing effects. Compared with products of the same kind, its outstanding advantages are also represented with excellent performance in processing meat products: It has the same antioxidation as VE; when used in combination with VC (AOB v.s. iso-ascorbate or iso-ascorbate sodium=0.01:10-10:0.01, preferably 0.0:10-10:0.1, by weight), by virtue of its capacity of clearing free NO-radicals(NO.) effectively, it may significantly reduce the use level of colorants (nitrates or nitrites), or on the precondition of maintaining the use level, it may significantly reduce the residue of these colorants, inhibit formation of N-nitrosamine, and thereby increase safety of meat products. For monascus pigment commonly used in meat products, AOB has antioxidant and color-protecting effects, which may help to increase its stability, improve color of meat products, and increase their commercial value. Meanwhile, due to presence of multi-hydroxy phenolic compounds, AOB has good water-maintaining and moistening performance, which may help to improve texture of meat products. This is quite important for Western sliced products.

With the characteristics of good quality, high safety, low cost, and natural, nutritional, multi-functional, but no peculiar smell, AOB is used in but not limited to the following aspects: edible oil (vegetable oil and fish oil, etc.), oil-containing food (mayonnaise, etc.), meat products (Western and Chinese products), aquatic products (shrimps, crabs, and fish, etc.), fruit juice, soft drinks (carbonated drinks, non-carbonated drinks, and tea drinks), brewed wine (grape wine, yellow wine, and beer), milk products (fresh milk and milk-containing drinks), condiments (oyster sauce, etc.), puffed food (oil-covering type), and cakes, etc. The suggested used level is 0.005-0.05% w/w.

AOB of this invention may be used in food systems alone or in combination with other natural anti-oxidant (such as phospholipid and phytic acid), vitamins [such as vitamin E (VE), vitamin C (VC) and their derivatives], metallic ion chelating agents (such EDTA and citric acid), and surfactants (such as Span 80 and Span 40), etc., resulting in significant synergistic effects.

After added to various food systems in appropriate proportion, AOB of this invention may remove active free oxygen-radicals, resist lipid peroxidation, prolong the shelf life, decrease the use level and residue of coloring agents (nitrates or nitrites), and exhibit multiple functions sucha as antibacterial, bacteriostatic, freshness-keeping, color-protecting, odor-removing, and taste-modifying effects.

If required, AOB of this invention may be manufactured in various forms, such as powder, aqueous solution, micro-emulsion, and micro-capsule. Its lipophilic property may also be improved by further structure modification (such as esterification of carbonide flavones with chloride palmitate).

This invention will be illustrated through the following non-restrictive examples. The amount of AOB used in the following examples are of w/w (weight/weight) percentage.

### EXAMPLE 1

Application of AOB in Western Meat Products:

During stuffing and mixing of Western sausage, add AOB (calculated in % w/w on the basis of meat stuffing, previously dissolved in water) in appropriate proportion, tea polyphenol as control. Comprehensively evaluate the antioxidation of AOB in Western meat products by the modified thiobarbituric acid (TBA) method in combination with chromatic aberration determination, texture profile, and nitrite content measurement. Optimal products were obtained when adding 0.03% AOB and decreasing the use level of nitrite and iso-ascorbate sodium to half of the original formula. That is to say, AOB thus used, on the one hand, can effectively delay lipid oxidation, inhibit formation of MDA and increase shelf life; on the other hand, it can significantly decrease the nitrite in finished products, and increase edible safety; meanwhile, it has not any adverse effects on flavor, color, and texture of sausage. In Western sausage, AOB has a stronger general effect than tea polyphenol, and has showed synergistic antioxidation with iso-ascorbate sodium.

### EXAMPLE 2

Application of AOB in Chinese Sausage:

Add AOB when mixing the meat stuffing (calculated in % w/w on the basis of meat stuffing, previously dissolved in water or alcohol). The experiment showed that: after adding 0.03% AOB on the basis of the original formula, the results of POV (peroxidation value) and AV (acid value) showed that the antioxidation of sausage were extremely increased; the nitrite content in finished products was only 56% of that in the control; when used in 0.03%, it had stronger effects than being used in 0.06%; when used in this amount (0.03%), it can significantly increase antioxidation of finished products, effectively clear away nitrites, and further block the formation of N-nitrosamine. Meanwhile, it does not have any adverse effects on the flavour, color and texture of the products and thus can be readily accepted by consumers.

### EXAMPLE 3

Application of AOB in Preserved Ham:

AOB was dissolved in water to produce a 0.03% solution, TBHQ (previously dissolved in ethanol) in the same concentration as control. A slice of Jinhua ham with a thickness of 1 cm was soaked for 2 min, and the effects of AOB on antioxidation and sensory quality of the slice of Jinhua ham were compared. AOB can mask flavor of fresh preserved ham to some extent; however, there were no differences in flavor and taste when the preserved ham was cooked; chromatic aberration determination showed that, there were no significant effects on color of ham (p>0.10); after stored in an incubation at 50±1° C. for 11 days, AV and POV measurements showed that, in the AOB and the TBHQ experimental groups compared with the control group, lipid oxidation was significantly inhibited and the shelf life was significantly prolonged. AOB had slightly stronger effects than TBHQ.

### EXAMPLE 4

Application of AOB in Aquatic Products:

Place the caught Macrobrachium rosenbergii and Chinese mitten-handed crabs in pond with clean water and maintain temporarily for 20-24 h (increase oxygen), and add 0.015% AOB in the water. While spitting residues in alimentary tract, shrimps and crabs take in certain amount of AOB antioxidant. After then, the shrimps and crabs were processed into soft cans, and normal-temperature, low-temperature storage experiment, and high-temperature oxidation experiment were conducted, respectively. AOB added to the temporary water significantly increased antioxidation of the products. The color-maintaining performance of finished products is dramatically better than that of the control (p<0.05). AOB effectively inhibits astacin (astaxanthin) color-fading oxidation and shrimp-head blackening oxidation. In the storage experiment at 35±1° C., bag-expanding rate was obviously decreased (p<0.05), demonstrating certain bacteriostatic, fressness-keeping effects. After the soft cans were stored for 3 months, the MIA content of shrimp or crab muscle homogenate was obviously lower than that of the blank control; shown as follows:

| | | OD₅₃₀ value at | | | |
|---|---|---|---|---|---|
| | | normal storage temperature | | | |
| Types | Groups | 0 | 30 d | 60 d | 90 d |
| Luo's pond | Blank-control | 0.018 | 0.065 | 0.099 | 0.137 |
| shrimps | AOP experimental | 0.011 | 0.037 | 0.064 | 0.096 |
| | group | | | | |
| China's | Blank-control | 0.021 | 0.073 | 0.111 | 0.148 |
| Eriocheir crabs | AOP experimental | 0.015 | 0.040 | 0.075 | 0.108 |
| | group | | | | |

### EXAMPLE 5

Application of AOB in Puffed Foods:

0.5 g AOB was dissolved in 10 mL 95% ethanol, mixed with 1000 g palm oil, sprayed to cake surface during oil-covering of puffed Xuemi cakes (oil-covering rate 18-20%, oil-covering temperature 60° C.), and packaged after cooled down. Place the sample in an incubation at 70±1° C. to accelerate oxidation; after certain time intervals, extract grease from the sample with Suo's extraction method, and determine MDA content with modified TBA method. AOB had a slightly stronger antioxidation than TBHQ, and obviously stronger than tea polyphenol.

### EXAMPLE 6

Application of AOB in Condiments:

Mayonnaise is an emulsified semi-solid food, a condiment with high nutrition, which is made mainly from egg yolk and edible vegetable oil, processed by adding a number of auxiliary materials. On the basis of basic formula (sunflower seed oil 70%, fresh egg yolk 14%, pure white vinegar 12%, sugar 2%, salt 1%, and dry mustard 1%), AOB was added in various proportions, meanwhile taking tea polyphenol (TP) as control and setting a blank control. The antioxidations of AOB and TP were compareed through sensory evaluation, chromatic aberration analysis, and POV and total carbonylated compounds (TCC) measurements. The results showed that, AOB had no significant effects on color and sensory quality of products. Place the sample at 45° C. to accelerate oxidation; POV and TCC showed that, the two antioxidants had certain effects in preventing lipid oxidation and prolonging mayonnaise shelf life. Here, 0.03% AOB had the best antioxidation; when rancidity occurred in the blank control group, the POV and TCC of the 0.03% AOB group were only 43.2% and 47.9% of those in the controll group; moreover, 0.03% AOB had stronger effects than those of TP at the same use level.

### EXAMPLE 7

Application of AOB in Milk Sterilized at High Temperature:

Before milk packaged in square paper-box were sterilized instantaneously at high temperature, AOB was added and controlled to be less than 75 mg/L. The results showed that AOB has no adverse effects on colloid system and sensory quality of milk, and it can obviously improve the anti-free radical activity and antioxidation of the products. When AOP was added in 75 mg/L, the anti-OH capacity of milk is 200% of that in the controll group, endowing products with a new health concept.

### EXAMPLE 8

Application of AOB in Fruit-Juice Drinks:

To pop-can orange drinks with intensified VC, AOB was added in 120 mg/L; The products thus produced had a unique cool taste with vivid and stable color as well as prolonged shelf life. At normal storage temperature, the control without AOB showed a darkened color in the 8^{th} month, with slight turbidity in its system, and the quality decreased; however, in the 12^{th} month, the sample with AOB maintained vivid orange-yellow color, stable and uniform system, and HPLC determination showed that the loss of its VC was just ½ of that in the controlled group. It showed that AOP can resist oxidation of orange juice, make anthocyanin stable, protecte VC, and it has good compatibility with orange-juice system.

### EXAMPLE 9

Application of AOB in Soft Drinks:

When used in soft drinks (including carbonated drinks, non-carbonated drinks, and tea drinks, etc.), AOB acts both as an antioxidant and a nutrition intensifier; the addition amount is controlled at 150-210 mg/L, and the use of sugar may be appropriately decreased. The product is mainly characterized by fragrance of bamboo leaves, enriched flavones functional factors, low calorie, stable quality, and its capacity of clearing heat, quenching thirst, soothing the sore throat, and inducing diuresis. Therefore, it is a new nutritional healthcare drink.

### EXAMPLE 10

Application of AOB in Brewed Wine:

When added to brewed wine (grape wine, yellow wine, and beer), AOB acts both as an antioxidant and a nutrition intensifier. The amount used is commonly controlled at 60-500 mg/L. It is added before wine base filtration and filling. Taking Shaoxing Ta-brand Jiafan Wine as an example, when the amount of AOB is 150 mg/L, the capacity of clearing O₂⁻ and OH, determined by chemiluminescence method, are increased by 40.0% and 28.5% compared with the original wine. When the same amount of AOB was added to beer, the chromaticity is slightly increased, the turbidity remain unchanged, and after heat stability test, the turbidity is obviously lower than that of the control; the rise of diacetyl content is obviously inhibited, and the antioxidation and storage stability are obviously increased. AOB has good compatibility with wine body of brewed wine. When controlled in a certain range of addition amount, AOB may maintain not only the original quality of wine body, but endow the product with a simple-but-elegant bamboo fragrance and mellow taste; as a natural, multi-functional biological antioxidant, AOB may be used in brewed wine for nutrition intensification and fresh-keeping.

### EXAMPLE 11

Application of AOB in Edible Oil:

10 g AOB was dissolves in 40 g Span40 (heated if needed). 50 g Span80 was added and mixed well to produce lipid-soluble AOB solution with mass fraction 0.10; when used, AOB was added according to the actual amount needed in the oil; in general, the addition amount of AOB in pure grease system (palm oil, soybean oil, sunflower seed oil, and fish oil, etc.) is 0.01-0.05%.

Obviously, this invention may have many modifications and changes in light of the above examples. Therefore, it should be understood that, in the scope of the attached claim, this invention may be implemented by other methods in addition to the methods described herein.

1. An antioxidant of bamboo leaves (AOB), wherein the chemical structures of its representative components are:

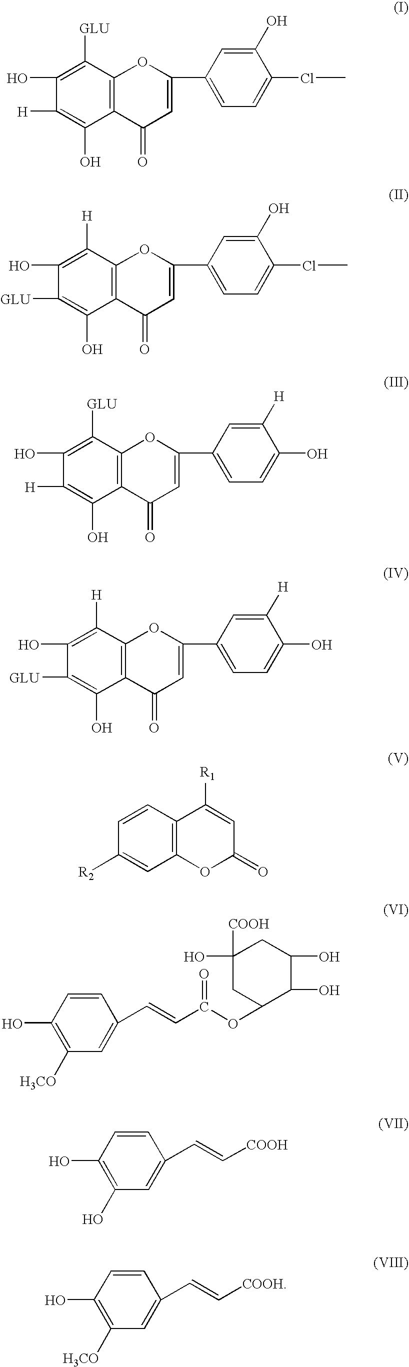

2. The antioxidant of bamboo leaves of claim 1, wherein said antioxidant is a complex mixture with synergistic effects, the appearance of the antioxidant is yellow or brown-yellow powders or particles; wherein the main active ingredient of the antioxidant are flavones, lactones, and phenolic acids, and the representative compounds includes Orientin (I), Homoorientin (II), Vitexin (III), Isovitexin (IV), Hydrxyl-Coumarin (V), Chlorogenic acid (VI), Caffeic acid (VII), and Ferulic acid (VIII).

3. The antioxidant of bamboo leaves of claim 2, wherein said antioxidant has ≧30% total flavones content when determined in aluminum nitrate-sodium nitrite colorimetry using rutin as reference; said antioxidant has ≧15% total lactones content when determined in iso-hydroximic acid colorimetry using aescine as reference; and said antioxidant has ≧7.5% phenolic acids content (total phenols−total flavones) when determined in forint reagent reduction-colorimetry using p-hydroxybenzoic acid as reference.

4. The antioxidant of bamboo leaves of claim 1, wherein the infrared chromatogram of said antioxidant which is tabletted with potassium bromide shows that, there are characteristic absorptions at or near 3400, 2935, 1626, 1080, and 616 cm⁻¹; when said antioxidant is dissolved in spectrum-pure methanol, the ultraviolet chromatogram shows that, there are two main absorption peaks in the range of 240-400 nm, including a strong absorption peak near 270 nm, and a secondary strong absorption peak near 330 nm.

5. A use of an Antioxidant of Bamboo Leaves as a natural, nutritional and multi-functional food additive in edible oil, oil-containing food, Chinese or Western meat products, aquatic products, fruit juice, milk products, soft drinks, brewed wine, condiments, puffed food, and cakes for the purpose of clearing away active free oxygen-radicals, lipid antioxidation, prolonging shelf life, decreasing the use level and residue of nitrate or nitrite colorants, antibacterial, bacteriostatic, keeping fresh, color-protecting, odor-removing, and taste-modifying, wherein the amount of addition of said antioxidant is in the range of 0.005-0.05% by weight.

6. A use of an Antioxidant of Bamboo Leaves as an additive for meat products to decrease the use level and residue of nitrate or nitrite, to inhibit formation of N-nitrosamine, to increase stability of monascus pigment, and/or to increase water-maintaining and moistening performance of meat products.

7. The use of claim 5 or claim 6 wherein said Antioxidant of Bamboo Leaves is used alone or in combination with other natural antioxidants, vitamins, metallic ion chelating agent, or surfactants.

8. The use of claim 7 wherein said natural antioxidants include phospholipid and phytic acid, said vitamins include VE, VC, and their derivatives, said metallic ion chelating agents include EDTA and citric acid, and said surfactants include Span80 and Span40.

9. The use of claim 5 or claim 6 wherein said Antioxidant of Bamboo Leaves is used in the form of powder, aqueous solution, micro-emulsion, or micro-capsule.

10. The use of claim 6 wherein the usage amount of said Antioxidant of Bamboo Leaves is 0.005-0.05% by weight of meat products, and said meat products comprises sausage, Chinese sausage, or preserved ham.
